(19) United States
(12) Patent Application Publication
(10) Pub. No.: US 2024/0358987 A1
(43) Pub. Date: Oct. 31, 2024

(54) Mechanical Clamshell Cathlock

(71) Applicant: Bard Peripheral Vascular, Inc., Franklin Lakes, NJ (US)

(72) Inventors: Christian Andersen, Queen Creek, AZ (US); Diana Fiumefreddo, Salt Lake City, UT (US); Ian N. Thomas, West Bountiful, UT (US); Jessica Hoye, Phoenix, AZ (US); Bryon Ray Densley, Rochester, MN (US); Luul H. Farah, Salt Lake City, UT (US)

(21) Appl. No.: 18/687,754

(22) PCT Filed: Sep. 1, 2021
(86) PCT No.: PCT/US21/48746

§ 371 Date: Feb. 28, 2024

(51) Int. Cl. A61M 39/04

(52) U.S. Cl. CPC A61M 39/04

(57) ABSTRACT

Embodiments disclosed herein are directed to a clamshell carhlock device and methods thereof. The cathlock includes a first portion hingedly coupled to a second portion and rotatable through a plane extending perpendicular to a central longitudinal axis. A user can apply opposing, radially inward, “pinching” force to transition the cathlock to the closed position, mitigating applying any pressure directly to the patient. The cathlock further includes a compliant sleeve coupled to an inner surface thereof and configured to slidably engage the cathlock with the catheter in both the open and closed positions. The sleeve can engage the catheter in an interference fit to allow a user to position the cathlock along the catheter and for the cathlock to remain in place until repositioned.

### SUMMARY

Briefly summarized, embodiments disclosed herein are directed to a “clamshell” cathlock device configured for coupling a catheter to a port stem. Proximally trimmable catheters allow for post-placement sizing of the catheter. When placing a catheter and port assembly, the position of the distal tip of the catheter can be important for the efficacy of the treatment. For example, when placing a catheter within the superior vena cava, if the distal tip of the catheter falls short of the target area, the efficacy of the medicament is reduced. If the distal tip is advanced too far, the distal tip can cause arrhythmia. The distance between the distal tip of the catheter and the port can vary since the distances between the target location, insertion site to the vasculature, and the location of the port can vary between patients and procedures. Estimating the catheter length before placement can lead to errors that result in misplacement of the distal tip.

Proximally trimmable catheters allow for placement of the catheter distal tip at the target location before trimming a proximal portion of the catheter to the correct length. The clinician can then attach the catheter to a subcutaneous port, or similar access device. However, securing the catheter to the port can be challenging. The connection must be leak-proof, especially under high-pressure infusion. Further, manipulating the catheter and port within the confined, wetted environment of a subcutaneous access site can lead to slippage, undue trauma to the access site, or misplacement of the catheter distal tip.

Disclosed herein is a cathlock system including, a catheter defining a lumen, a stem configured to engage the lumen of the catheter, and a cathlock configured to engage an outer surface of the catheter and secure the catheter to the stem, the cathlock including a body having a first portion hingedly coupled to a second portion and transitionable between an open position and a first closed position, a locking mechanism configured to releasably engage the first portion and the second portion in the first closed position, and a sleeve coupled to an inner surface the first portion or an inner surface of the second portion and configured to secure the cathlock to the catheter in both the open position and the first closed position.

In some embodiments, the body in the first closed position defines a channel having a diameter that is less than an outer diameter of the catheter. In some embodiments, the sleeve defines a lumen having a first inner diameter that is equal to the outer diameter of the catheter and is elastically deformable to a second inner diameter that is less than the first inner diameter. In some embodiments, the sleeve is elastically deformable to a third inner diameter that is larger than the first inner diameter. In some embodiments, the sleeve is formed of a plastic, polymer, elastomer, rubber, or silicone rubber.

In some embodiments, the body further includes a second closed position, a diameter of the channel in the second closed position being larger than the outer diameter of the catheter. In some embodiments, the cathlock system further includes a latching mechanism configured to provide mechanical advantage to transition from the second closed position to the first closed position. In some embodiments, one or both of the first portion and the second portion are formed of a rigid or resilient material. In some embodiments, the sleeve is slidably engaged with the catheter in the open position. In some embodiments, the sleeve engages the catheter in the open position in an interference fit and is configured to be positioned on the catheter at a first longitudinal position and remain at the first longitudinal position until repositioned to a second longitudinal position, different from the first longitudinal position. In some embodiments, the stem is in fluid communication with a port.

Also disclosed is a method of coupling a catheter to a stem including, slidably engaging a catheter with a sleeve of a cathlock, the sleeve coupled to an inner surface of a first portion of a cathlock body that is hingedly coupled to a second portion of the cathlock body, the first portion and the second portion being in an open position, urging a proximal end of the catheter onto a stem, sliding the sleeve longitudinally along the catheter to align the cathlock with a proximal portion of the catheter, and rotating the first portion relative to the second portion to transition the cathlock from the open position to a closed position.

In some embodiments, rotating the first portion relative to the second portion further includes applying a first radially inward force to the first portion and a second radially inward force, opposite the first radially inward force, to the second portion. In some embodiments, the cathlock body in the closed position defines a channel having a diameter that is equal to or less than an outer diameter of the catheter. In some embodiments, the sleeve defines a channel having a first inner diameter that is equal to the outer diameter of the catheter and is elastically deformable to a second inner diameter that is less than the first inner diameter.

In some embodiments, the sleeve is elastically deformable to a third inner diameter that is larger than the first inner diameter. In some embodiments, the sleeve is formed of a plastic, polymer, elastomer, rubber, or silicone rubber. In some embodiments, the method further includes engaging a latching mechanism to couple the first portion and the second portion in a second closed position, the cathlock body defining a second diameter in the second closed position that is larger than the outer diameter of the catheter.

In some embodiments, the method further includes engaging the latching mechanism to transition the cathlock body from the second closed position to the first closed position. In some embodiments, one or both of the first portion and the second portion are formed of a rigid or resilient material. In some embodiments, the sleeve slidably engages the catheter in the open position. In some embodiments, the method further includes sliding the cathlock in the open position along the catheter to a first longitudinal position, the sleeve engaging the catheter in an interference fit to maintain the cathlock at the first longitudinal position, and sliding the cathlock to a second longitudinal position different from the first longitudinal position.

Also disclosed is a catheter locking system including, a catheter defining a lumen, a port including a stem configured to engage the lumen of the catheter, and a cathlock configured to engage an outer surface of the catheter and secure the catheter to the stem, the cathlock having, a body having a first portion hingedly coupled to a second portion and transitionable between an open position and a first closed position, a locking mechanism configured to releasably engage the first portion and the second portion in the first closed position, and a sleeve coupled to one of the first portion or the second portion and configured to couple the cathlock to the catheter in both the open position and the first closed position.

In some embodiments, the body in the first closed position defines a channel having a diameter that is less than an outer diameter of the catheter. In some embodiments, the sleeve defines a lumen having a first inner diameter that is equal to the outer diameter of the catheter and is elastically deformable to a second inner diameter that is less than the first inner diameter. In some embodiments, the sleeve is elastically deformable to a third inner diameter that is larger than the first inner diameter. In some embodiments, the sleeve is formed of a plastic, polymer, elastomer, rubber, or silicone rubber.

In some embodiments, the body further includes a second closed position, a diameter of the channel in the second closed position being larger than the outer diameter of the catheter. In some embodiments, the catheter locking system further includes a latching mechanism configured to provide mechanical advantage to transition from the second closed position to the first closed position. In some embodiments, one or both of the first portion and the second portion are formed of a rigid or resilient material. In some embodiments, the sleeve is slidably engaged with the catheter in the open position. In some embodiments, the sleeve engages the catheter in the open position in an interference fit and is configured to be positioned on the catheter at a first longitudinal position and remain at the first longitudinal position until repositioned to a second longitudinal position, different from the first longitudinal position.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 shows a perspective view of a clamshell cathlock in an exemplary environment of use, in accordance with embodiments disclosed herein.

FIG. 2 shows a perspective view of a clamshell cathlock in an open position, in accordance with embodiments disclosed herein.

FIG. 3 shows a perspective view of a clamshell cathlock in a closed position, in accordance with embodiments disclosed herein.

FIGS. 4A-4D show an exemplary method of use for a clamshell cathlock, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

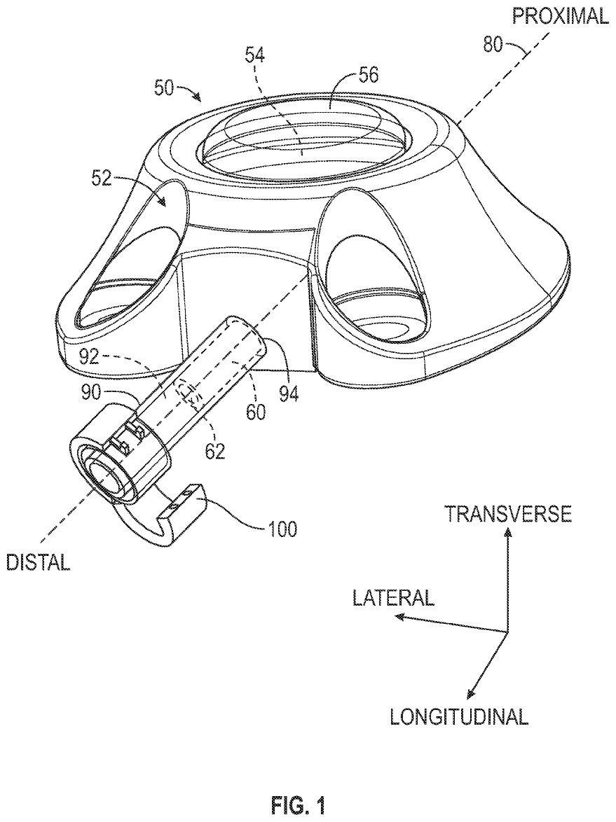

To assist in the description of embodiments described herein, as shown in FIGS. 1-3, a longitudinal axis extends substantially parallel to an axial length of the catheter. A lateral axis extends normal to the longitudinal axis, and a transverse axis extends normal to both the longitudinal and lateral axes. As used herein, a horizontal plane extends along the lateral and longitudinal axes. A vertical plane extends normal to the horizontal plane.

Unless defined otherwise, all technical and scientific terms used herein have the same meaning as commonly understood by those of ordinary skill in the art.

Embodiments described herein are directed to a clamshell cathlock (“cathlock”) 100 configured to secure a catheter 90 with a stem 60, or similar rigid structure. FIG. 1 shows a clamshell cathlock 100 in an exemplary environment of use, for example, coupling a catheter 90 or similar compliant tube, with a stem 60 of a port 50. As will be appreciated, the port 50 is an exemplary medical device and embodiments disclosed herein can be used with various medical devices that require coupling a compliant tube with a rigid stem to provide fluid communication therebetween.

In an embodiment, the port 50 can generally include a body 52 defining a reservoir 54 that is in fluid communication with a lumen 62 of a stem 60. The port 50 can further include a needle penetrable septum 56 disposed over the reservoir 54 and configured to provide access thereto. In use, an access needle can extend percutaneously, through the needle-penetrable septum 56 and into the reservoir 54 to provide fluid communication therewith.

In an embodiment, the catheter 90 can include an elongate body extending longitudinally between a distal tip and a proximal end 94 and can define a catheter lumen 92. In an embodiment, the distal tip of the catheter 90 can be disposed within a vasculature of the patient to provide fluid communication therewith. In an embodiment, the catheter 90 can be urged axially over the stem 60 to engage the stem 60 in an interference fit and provide fluid communication between the catheter lumen 92 and the stem lumen 62. In an embodiment, the catheter 90 can be formed of a compliant material such as a plastic, polymer, polyurethane, elastomer, composite, rubber, silicone rubber, or the like. In an embodiment, the catheter 90 can be configured to elastically deform and can be stretched over the stem 60 to engage the stem 60 in an interference fit and provide a fluid tight seal therebetween.

In an embodiment, a cathlock 100 can be coupled to an outer surface of the catheter 90 and compress the catheter 90 onto the stem 60 to further secure the catheter 90 thereto. The cathlock 100 can be of particular importance to ensure the catheter 90 does not disengage the stem 60 accidentally. Further, the cathlock 100 can secure the catheter 90 to the stem 60 to allow for increased pressure or increased flow rate therethrough without failure or leakage from the catheter 100/stem 60 connection.

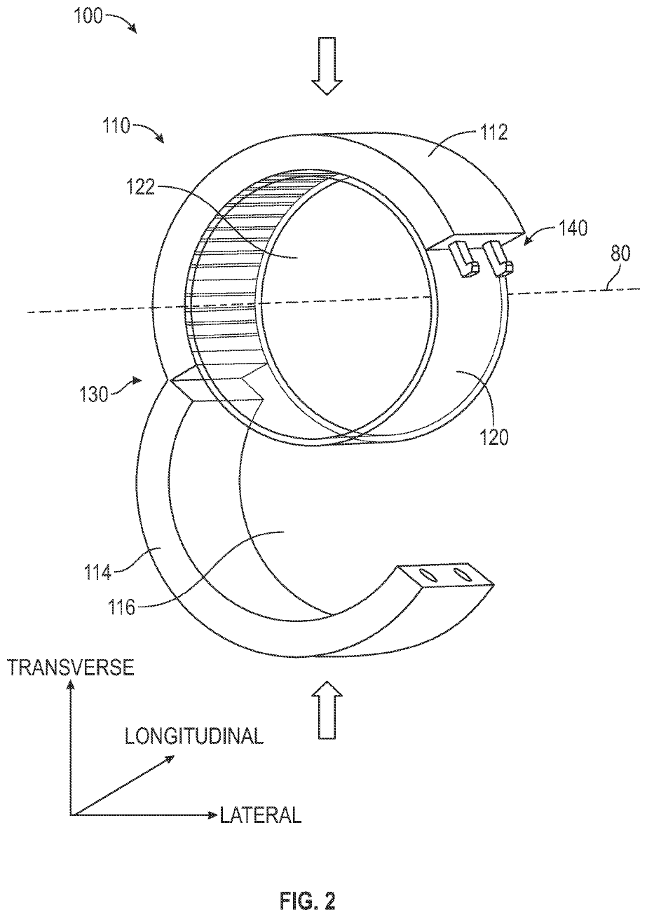

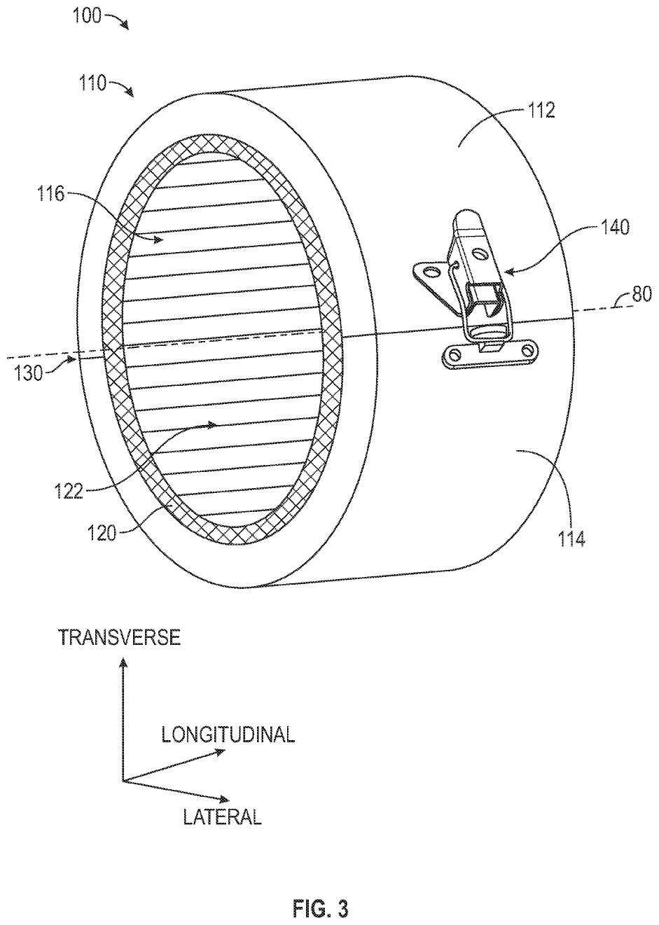

In an embodiment, as shown in FIGS. 2-3, a cathlock 100 can include a body 110 having a first portion 112 hingedly coupled to a second portion 114 with a hinge 130. The hinge 130 can be a mechanical hinge, living hinge, or similar structure configured to allow the first portion 112 and second portion 114 to rotate relative to each other. In an embodiment, the first portion 112 and second portion 114 can rotate relative to each other between an open position (FIG. 2) and a closed position (FIG. 3). The cathlock 100 can further include a latch 140 or similar mechanism configured to secure the body 110 in one or more closed positions. Exemplary latching mechanisms 140 can include clip, barb, detent, ties, sutures, ratchet and pawl, zip tie, over-center latch, combinations thereof, or the like. In an embodiment, the body 110 can be formed of a first material providing substantially rigid or resilient mechanical properties. The body 110 can be formed of a plastic, polymer, metal, alloy, Nitinol, composite, combinations thereof, or the like.

In an embodiment, the cathlock body 110 in the closed position (FIG. 3) can define a channel 116 configured to receive one or both of a portion of the catheter 90 and the stem 60 therethrough. In an embodiment, a diameter of the channel 116 can be equal to or less than an outer diameter of the catheter 90. In an embodiment, a diameter of the channel 116 can be equal to or less than an outer diameter of a portion of the catheter 90 disposed over the stem 60.

In an embodiment, the first portion 112 and the second portion 114 can rotate relative to each other through a plane extending perpendicular to a central longitudinal axis 80 between an open position and one or more closed positions. Advantageously, as shown in FIG. 2, the cathlock 100 can be transitioned from the open configuration to the closed configuration by applying opposing radially inward forces, e.g. a pinching force, perpendicular to the central longitudinal axis 80. As such, applying a opposing “pinching” forces mitigates applying any pressure directly to the patient which may cause discomfort. Further, the opposing forces provide more control to the user when applying pressure to the catheter 90/port 50/cathlock 100 assembly and mitigates slippage and undue trauma within the confined wetted environment of the subcutaneously placement site.

In an embodiment, the cathlock 100 can further include a sleeve 120. The sleeve 120 can be formed of a second material providing flexible, compliant, or elastically deformable mechanical properties. In an embodiment, the sleeve 120 can be formed of a plastic, polymer, elastomer, rubber, silicone rubber, composite, combinations thereof, or the like. In an embodiment, the sleeve 120 can be coupled to an inner surface of one of the first portion 112 or the second portion 114 using adhesive, bonding, welding, fasteners, combinations thereof, or the like. In an embodiment, the sleeve 120 can define a lumen 122 configured to receive a portion of the catheter 90 therethrough and slidably engage the cathlock 100 with the catheter 90.

In an embodiment, as shown in FIG. 2, the sleeve 120 in an unstressed state can define a substantially circular lumen 122. To note, as shown in FIGS. 1, 2 and 4A, the sleeve 120 is shown in wire frame for clarity. In an embodiment, a diameter of the sleeve lumen 122 in an unstressed state can be greater than an outer diameter of a portion of the catheter 90. As such, the sleeve 120 can slidably engage the catheter 90. In an embodiment, a diameter of the sleeve lumen 122 in an unstressed state can be equal to, or less than an outer diameter of a portion of the catheter 90. Advantageously, the sleeve 120 can slidably engage the catheter 90 in an interference fit. As such, in use, a clinician can thread the catheter 90 through the sleeve lumen 122 to slidably engage the catheter 90 therewith. The clinician can position the cathlock 100 at a first longitudinal position on the catheter 90. The sleeve 120 can engage the catheter 90 in an interference fit to maintain the position of the cathlock 100 at the first longitudinal position relative to the catheter 90 until the cathlock 100 is repositioned by the user to a second longitudinal position.

Advantageously, the sleeve 120 can prevent the cathlock 100 from sliding off of the catheter 90 and touching a floor surface or similar surface. Advantageously, the sleeve 120 can be formed of a compliant material and can be compressible between the catheter 90 and the cathlock 100 to provide increased grip therebetween. Advantageously, the second material of the sleeve 120 can provide an increased frictional co-efficient to provide increased grip between the cathlock 100 and the catheter 90. This can be of particular importance in the naturally wetted environment of subcutaneous placement.

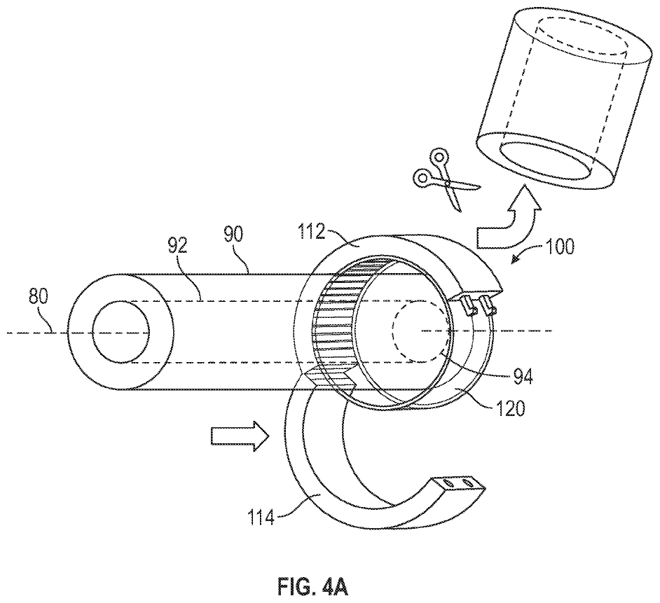

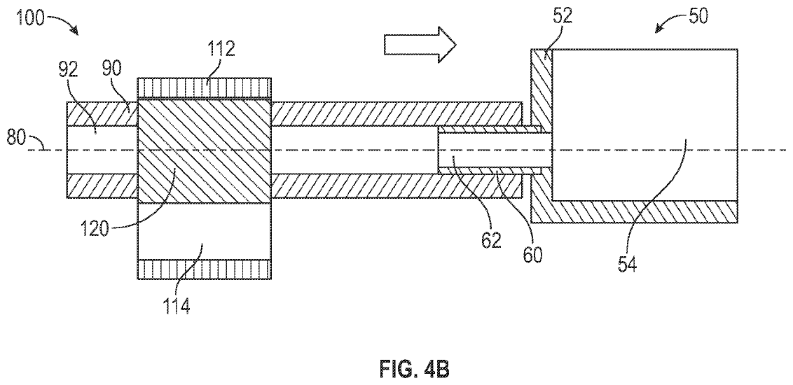

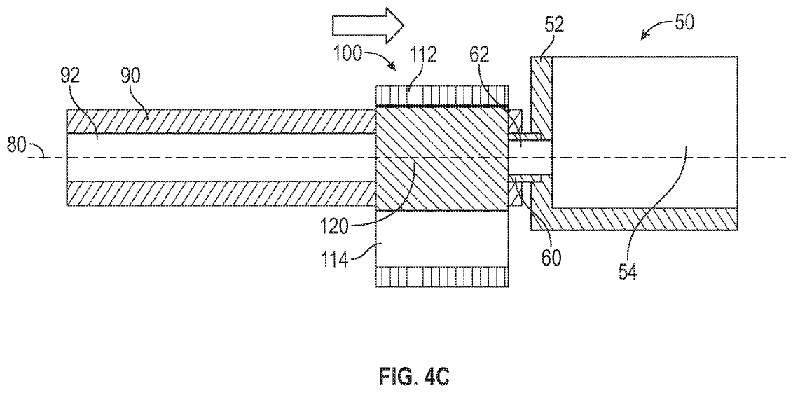

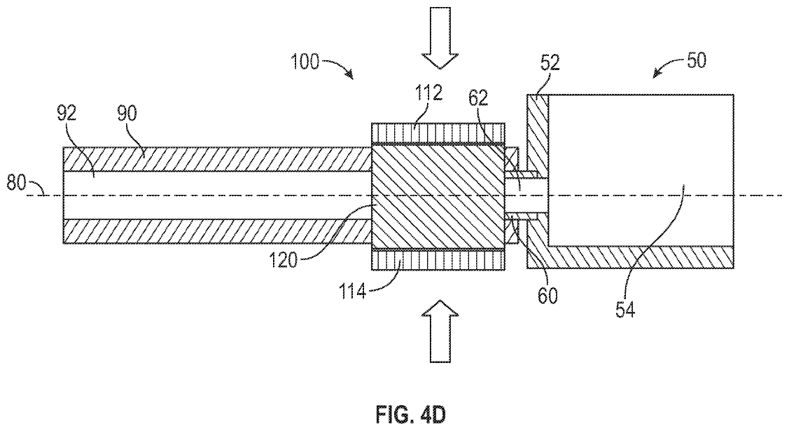

In an exemplary method of use, as shown in FIGS. 4A-4D, a cathlock 100 is provided as described herein. Initially, a distal end of the catheter 90 can be placed at a target location within a vasculature of the patient. Optionally, as shown in FIG. 4A, a proximal end 94 the catheter 90 can be trimmed to a suitable length. The proximal end 94 of the catheter 90 can be fed through the lumen 122 of the sleeve 120 to slidably engage the cathlock 100 with the catheter 90 while the cathlock 100 is in the open position.

In an embodiment, as shown in FIGS. 4B-4C, the cathlock 100 can engage the catheter 90 in an interference fit. As such a user can position the cathlock 100 at a first position along a longitudinal length of the catheter 90 and the cathlock 100 can remain in place until repositioned by the user. The user can then urge the proximal end 94 over the stem 60. The user can then reposition the cathlock 100 to longitudinally align with a portion of the stem 60, a proximal portion of the catheter 90, or both.

As shown in FIG. 4D, the user can then transition the cathlock 100 from the open position to a first closed position by applying opposing radially inward forces along an axis extending perpendicular to the central longitudinal axis 80 of the catheter 90. For example, a first force of the opposing forces can be applied to the first portion 112 of the cathlock body 110, and a second force of the opposing forces can be applied to the second portion 114 of the cathlock body 110 causing the first portion 112 and the second portion 114 to rotate relative to each other.

The latching mechanism 140 can engage the first portion 112 and the second portion 114 and maintain the cathlock body 110 in the first closed position. Optionally, the latching mechanism 140 can be configured to selectively disengage one of the first portion 112 or the second portion 114 allowing the cathlock body 110 to transition from the closed position to the open position.

In an embodiment, as shown in FIG. 3, the latching mechanism 140 can be an over-center latching mechanism 140 configured to couple the first portion 112 to the second portion 114 in a second closed position. A lever of the latching mechanism 140 can be configured to provide mechanical advantage to then transition the first portion 112 and the second portion 114 to the first closed position and maintain the cathlock body 110 in the first closed position until selectively released. In an embodiment, a diameter of the channel 116 in the first closed position can be less than a diameter of the channel in the second closed position.

In an embodiment, a diameter of the channel 116 or a diameter of the sleeve lumen 112, or both can be equal to or less than an outer diameter of the catheter 90. In an embodiment, a diameter of the channel 116 or a diameter of the sleeve lumen 122 or both can be equal to or less than an outer diameter of a portion of the catheter 90 disposed over the stem 60. As such, in the closed position, the cathlock 100 can compress the catheter 90 on to the stem 60 providing a secure, fluid-tight seal therebetween.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

The invention claimed is:

1. A cathlock system, comprising:
a catheter defining a lumen;
a stem configured to engage the lumen of the catheter; and
a cathlock configured to engage an outer surface of the catheter and secure the catheter to the stem, the cathlock comprising:
a body having a first portion hingedly coupled to a second portion and transitionable between an open position and a first closed position;
a locking mechanism configured to releasably engage the first portion and the second portion in the first closed position; and
a sleeve coupled to an inner surface the first portion or an inner surface of the second portion and configured to secure the cathlock to the catheter in both the open position and the first closed position.

2. The cathlock system according to claim 1, wherein the body in the first closed position defines a channel having a diameter that is less than an outer diameter of the catheter.

3. The cathlock system according to claim 1, wherein the sleeve defines a lumen having a first inner diameter that is equal to the outer diameter of the catheter and is elastically deformable to a second inner diameter that is less than the first inner diameter.

4. The cathlock system according to claim 3, wherein the sleeve is elastically deformable to a third inner diameter that is larger than the first inner diameter.

5. The cathlock system according to claim 1, wherein the sleeve is formed of a plastic, polymer, elastomer, rubber, or silicone rubber.

6. The cathlock system according to claim 1, wherein the body further includes a second closed position, a diameter of the channel in the second closed position being larger than the outer diameter of the catheter.

7. The cathlock system according to claim 6, further including a latching mechanism configured to provide mechanical advantage to transition from the second closed position to the first closed position.

8. The cathlock system according to claim 1, wherein one or both of the first portion and the second portion are formed of a rigid or resilient material.

9. The cathlock system according to claim 1, wherein the sleeve is slidably engaged with the catheter in the open position.

10. The cathlock system according to claim 1, wherein the sleeve engages the catheter in the open position in an interference fit and is configured to be positioned on the catheter at a first longitudinal position and remain at the first longitudinal position until repositioned to a second longitudinal position, different from the first longitudinal position.

11. The cathlock system according to claim 1, wherein the stem is in fluid communication with a port.

12-22. (canceled)

23. A catheter locking system, comprising:
a catheter defining a lumen;
a port including a stem configured to engage the lumen of the catheter; and
a cathlock configured to engage an outer surface of the catheter and secure the catheter to the stem, the cathlock comprising:
a body having a first portion hingedly coupled to a second portion and transitionable between an open position and a first closed position;
a locking mechanism configured to releasably engage the first portion and the second portion in the first closed position; and
a sleeve coupled to one of the first portion or the second portion and configured to couple the cathlock to the catheter in both the open position and the first closed position.

24. The catheter locking system according to claim 23, wherein the body in the first closed position defines a channel having a diameter that is less than an outer diameter of the catheter.

25. The catheter locking system according to claim 23, wherein the sleeve defines a lumen having a first inner diameter that is equal to the outer diameter of the catheter and is elastically deformable to a second inner diameter that is less than the first inner diameter.

26. The catheter locking system according to claim 25, wherein the sleeve is elastically deformable to a third inner diameter that is larger than the first inner diameter.

27. The catheter locking system according to claim 23, wherein the sleeve is formed of a plastic, polymer, elastomer, rubber, or silicone rubber.

28. The catheter locking system according to claim 23, wherein the body further includes a second closed position, a diameter of the channel in the second closed position being larger than the outer diameter of the catheter.

29. The catheter locking system according to claim 28, further including a latching mechanism configured to provide mechanical advantage to transition from the second closed position to the first closed position.

30. The catheter locking system according to claim 23, wherein one or both of the first portion and the second portion are formed of a rigid or resilient material.

31. The catheter locking system according to claim 23, wherein the sleeve is slidably engaged with the catheter in the open position.

32. The catheter locking system according to claim 23, wherein the sleeve engages the catheter in the open position in an interference fit and is configured to be positioned on the catheter at a first longitudinal position and remain at the first longitudinal position until repositioned to a second longitudinal position, different from the first longitudinal position.
